(19) United States
(12) Patent Application Publication
(10) Pub. No.: US 2021/0116369 A1
(43) Pub. Date: Apr. 22, 2021

(54) MEASUREMENT OF MOLECULAR INTERACTIONS AT SINGLE MOLECULE LEVEL USING SUBSTRATES THAT ENHANCE FLUORESCENCE DETECTION

(71) Applicant: BIO-RAD LABORATORIES, INC., Hercules, CA (US)

(72) Inventors: EVAN THRUSH, SAN ANSELMO, CA (US); NING LIU, WALNUT CREEK, CA (US); CARL MARLOWE, SAN FRANCISCO, CA (US); WILLIAM BRADLEY STRONG, EL CERRITO, CA (US)

(21) Appl. No.: 17/077,643

(22) Filed: Oct. 22, 2020

Related U.S. Application Data
(60) Provisional application No. 62/924,337, filed on Oct. 22, 2019.

(51) Int. Cl. G01N 21/552; G06T 7/00

(52) U.S. Cl. CPC G01N 21/553; G01N 33/68; G06T 7/0014

(57) ABSTRACT

The invention relates to methods for characterizing the binding interactions between binding partners. The invention pertains to methods comprising contacting a first binding partner with a second binding partner that exhibits fast-off rate binding characteristics with the first binding partner to generate the binding interaction between the first binding partner and the second binding partner, wherein the first binding partner is immobilized onto a substrate that is designed to enhance the fluorescence signal of fluorescent molecules located near the substrate's surface, and wherein the second binding partner is a molecule that emits a fluorescent signal or is conjugated to molecule that emits a fluorescent signal. The interactions between the two binding partners can be analyzed based on multiple transient interactions between the two binding partners.

### CROSS-REFERENCE TO RELATED APPLICATION

This application claims the benefit of U.S. Provisional Application 62/924,337 filed on Oct. 22, 2019 which is hereby incorporated by reference in its entirety.

### BACKGROUND

Developing substrates that enhance the fluorescence signal of fluorescent molecules located near the substrate's surface is desirable. Fluorescence enhancements of several orders of magnitude have been demonstrated. Typically, substrates used for the enhancement of fluorescence typically utilize surface resonances (i.e., plasmons) that occur at metal dielectric interfaces. Conventional prism based Surface Plasmon Resonance architectures can be used for this purpose. Metallic grating structures, photonic crystal structures, nano-patterned surfaces, nano-structured surfaces, etc. can also be used to enhance fluorescence.

Single Molecule Kinetic Fingerprinting (SMKF) provides binding characteristics of a molecule with its binding partner. Certain such methods of SMKF are disclosed in PCT Application Publication No. WO 2018/140157, which is incorporated herein by reference in its entirety. SMKF could be used for characterizing binding characteristics between binding members at a single molecule level. SMKF gives much more information about the binding interactions between binding partners, which could be used to reduce background/noise and improve sensitivity and specificity.

### BRIEF SUMMARY

The invention relates to methods for detecting and analyzing the binding characteristics of binding partners. Particularly, certain embodiments of the invention provide methods of detecting and analyzing the binding characteristics, for example, producing SMKF, of the binding interactions between a first binding partner and a second binding partner, wherein the first binding partner is immobilized onto a substrate that is designed to enhance a fluorescent signal emitted by molecules located near the substrate's surface and the second binding partner is a molecule that emits the fluorescent signal or is conjugated to molecule that emits a fluorescent signal, and wherein the second binding partner exhibits fast-off rate binding with the first binding partner.

In preferred embodiments, the invention provides methods comprising contacting a first binding partner with a second binding partner that exhibits fast-off rate binding with the first binding partner to generate the binding interaction between the first binding partner and the second binding partner and detecting the SMKF of the binding interactions between the first binding partner and the second binding partner by single molecule detection of the second binding partner, wherein the first binding partner is immobilized onto a substrate that is designed to enhance the fluorescence signal of fluorescent molecules located near the substrate's surface, and wherein the second binding partner is a molecule that emits fluorescent signal or is conjugated to molecule that emits a fluorescent signal.

The interactions between the two binding partners can be analyzed by measuring/determining transient interactions between the two binding partners.

### BRIEF DESCRIPTION OF DRAWINGS

FIG. 1. Schematic representation of certain embodiments of the invention.

FIG. 2. An example of a substrate that is designed to enhance the fluorescence signal of fluorescent molecules located near the substrate surface.

### DETAILED DESCRIPTION

As used herein, the singular forms “a”, “an” and “the” are intended to include the plural forms as well, unless the context clearly indicates otherwise. To the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Typically, “about” can mean a range of up to 0-10% of a given value.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

As use herein, the term “specific binding” between two molecules refers to measurable and reproducible binding between the two molecules based on specific interactions between the two molecules. Such specific interactions may include hydrogen bonding, hydrophobic forces, and ionic bonding. “Specific binding” does not necessarily require (although it can include) exclusive binding. For example, an antibody specifically binds to an antigen, typically, with the equilibrium dissociation constant (K_{D}) of lower than about 10⁻⁶ M, lower than about 10⁻⁹ M, or lower than about 10⁻¹² M.

On the other hand, “non-specific binding” between two molecules refers to the binding that is not based on specific interactions between the two molecules. Non-specific binding may result from non-specific interactions, such as Van Der Waals forces. K_{D} for the non-specific binding between two molecules is typically higher than about 10⁻⁵ M, higher than about 10⁻⁴ M or higher than about 10⁻² M. Sometimes non-specific interactions can't be characterized by traditional binding characteristics and can't be represented by a simple association/dissociation constant.

The term “target” as used herein includes an analyte of interest. A target can be a protein, lipid, nucleic acid (for example, RNA, DNA, locked nucleic acid (LNA), xeno nucleic acid (XNA)), sugar, and carbohydrate. Additional examples of targets are known in the art and such embodiments are within the purview of the invention.

The term “probe” is used to describe certain binding partners. Probe refers to a molecule that specifically binds to a target. Thus, a probe can be used to determine the presence of a target. Typically, a probe comprises a binding portion, which is involved in specifically bindings with its target, and a label portion, which provides a fluorescent signal that facilitates detection of the target. Thus, a probe is a molecule that emits a fluorescent signal or is conjugated to a molecule that emits a fluorescent signal. A fluorescent signal can be detected by a photosensitive device. Additional details of a fluorescent signal and molecules that emit a fluorescent signal are discussed below under the discussion of a “label”. Type of probes used in a specific application depends on the target molecules, available detection methods, specific substrate used, and other parameters that are well known in the art. Such embodiments are within the purview of the invention.

A “label” is a part of the probe or is conjugated to the probe and provides a fluorescent signal that is detectable a photosensitive device.

Exemplary fluorophores include, but are not limited to, Alexa dyes (e.g., Alexa 350, Alexa 430, Alexa 488, etc.), AMCA, BODIPY 630/650, BODIPY 650/665, BODIPY-FL, BODIPY-R6G, BODIPY-TMR, BODIPY-TRX, Cascade Blue, Cy2, Cy3, Cy5, Cy5.5, Cy7, Cy7.5, Dylight dyes (Dylight405, Dylight488, Dylight549, Dylight550, Dylight 649, Dylight680, Dylight750, Dylight800), 6-FAM, fluorescein, FITC, HEX, 6-JOE, Oregon Green 488, Oregon Green 500, Oregon Green 514, Pacific Blue, REG, Rhodamine Green, Rhodamine Red, ROX, R-Phycoerythrin (R-PE), Starbright Blue Dyes (e.g., Starbright Blue 520, Starbright Blue 700), TAMRA, TET, Tetramethylrhodamine, Texas Red, and TRITC. Additional labels that can be used in the probes in the methods disclosed herein are known and are described in the art, for example, in the PCT Publication Number WO 2018/140157, particularly, in paragraphs [0048] to [0053].

The binding portion and the label portion of the probe can be present in a single entity (e.g., a fluorescent molecule capable of binding a target) or multiple conjugated entities (e.g., a fluorescent molecule is conjugated to a molecule that can specifically bind to a target). Non-limiting examples of probes include natural or modified peptides, proteins (e.g., antibodies, affibodies, or aptamers), nucleic acids (e.g., polynucleotides, DNA, or RNA); polysaccharides (e.g., lectins, sugars), lipids, enzymes, enzyme substrates or inhibitors, ligands, receptors, antigens, haptens, small molecules, and synthetic nucleic acids such as LNA and XNA. Additional examples of probes suitable for use in the methods described herein are known and such embodiments are within the purview of the invention.

The binding between a probe and a label can also be identified via fluorescence resonance energy transfer (FRET). Typically, during FRET analysis a donor fluorophore in an excited electronic state transfers its excitation energy to an acceptor chromophore located in close proximity, for example, within about 1 to 15 nanometers of each other. Such proximity between a probe and a target is achieved in a specific binding interaction and, therefore, transfer of excitation energy from a probe conjugated to a donor fluorophore to an acceptor fluorophore conjugated to a target indicates binding between the probe and the target. Accordingly, in such embodiments, a probe or a target can be conjugated with a donor chromophore and a corresponding target or a probe can be conjugated with an acceptor chromophore and the binding between the probe and the target can be detected based on the emission of a fluorescent signal. Appropriate pairs of donor and acceptor fluorophores are known in the art and such embodiments are within the purview of the invention.

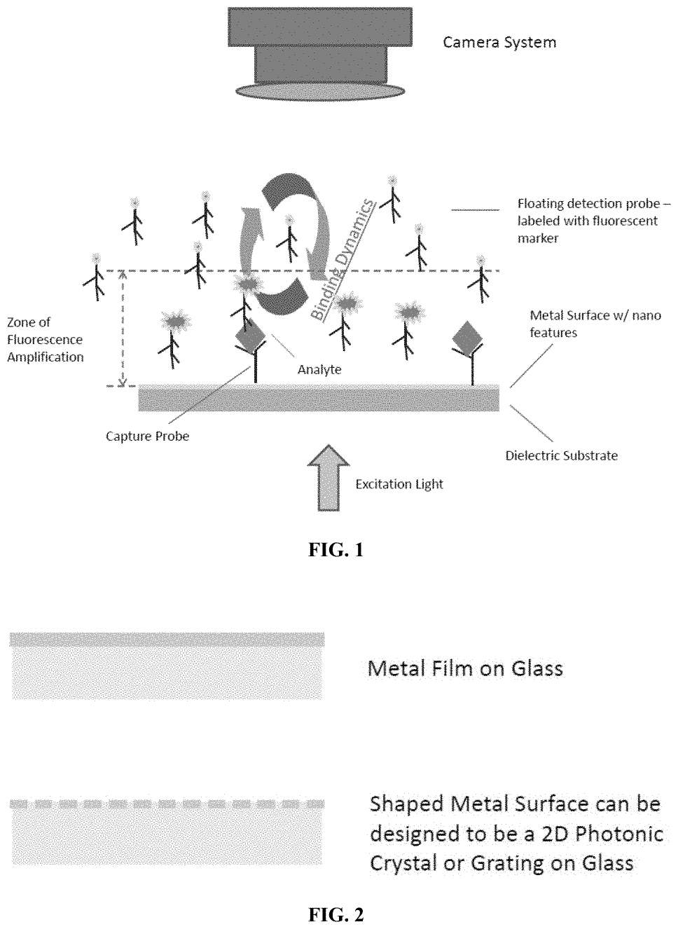

The term “kinetic fingerprinting” as used herein refers to the time dependent binding characteristics of probes with targets. The kinetic fingerprinting could be used to distinctly distinguish the specific binding between probes and targets from non-specific binding between probes and non-targets. For example, kinetic fingerprinting could be used to identify and distinguish signals that arise from binding of a probe to a target from the signals that arise from non-specific binding of a probe to a non-target. This is because a probe behaves in a unique way in its interactions with the corresponding target and if this behavior is observed between a probe and a target for a period of time, for example, over 5 to 10 minutes, then this unique behavior could be used to identify specific binding between the probe and the target. On the other hand, if the unique behavior between a probe and a target is not observed for a period of time, for example, over 5 to 10 minutes, then the lack of the unique behavior can be used to identify the binding between the probe and the target as non-specific. FIGS. 1A-1C and paragraphs [0025] to [0027] of the PCT publication WO2018/140157 describe certain aspects of the unique behaviors of the specific binding between a probe and its target and distinguishes such unique behavior from non-specific binding between a probe and a non-target. For a known pair of a probe and its specific target, this unique behavior can also be used to separate the unwanted signals that likely arise from non-specific interactions and, thus, the signals that would otherwise interfere with the measurement of specific binding interactions could be filtered or ignored during further analysis.

k_{off} is the first-order rate constant for the dissociation of the protein-ligand complex. The dimension of k_{off} is time⁻¹. 1/k_{off} refers to the “average lifetime” of the complex between binding partners. Some complexes dissociate quicker and some slower. Log 2/k_{off} refers to the half-life of the complex which is the time required for half of the molecules in complex to dissociate. A higher value for k_{off} means a shorter lifetime for the complex and vice versa. In an exemplary analysis, “residence time” of a probe on a target is proportional to the inverse of k_{off} The residence time of the binding is different from how long an experimental set up may be operated which may include many binding events with differing residence time.

Any two molecules can bind to one another with different affinities or may not bind to each other at all. The phrase “binding partners” as used herein describes molecules that specifically bind to each other. For example, one of the pair of the binding partners can be a probe and the other one can be a specific target molecule. Similarly, one of the binding partners can be an antibody and the other can be a specific antigen to which the antibody specifically binds. Examples of suitable binding partners include, but are not limited to, nucleic acid and nucleic acid; protein or peptide and nucleic acid; protein or peptide and protein or peptide; antigens and antibodies; receptors and ligands, haptens and cognate antibodies, polysaccharides and proteins, and pharmaceutical compounds and cognate biological targets. Additional examples of binding partners are known in the art and such embodiments are within the purview of the invention.

In molecular binding interactions, specificity indicates the degree to which a molecule recognizes its binding partner. For example, specificity of an immune system towards an antigen determines the degree to which the immune system can differentiate between different antigens and only respond to the relevant antigen. Thus, specificity defines the degree to which an immune response discriminates between antigenic variants. One can measure specificity of an immune apparatus, such as antibodies or T cells, by measuring the relative binding affinities of the antibodies or T cells for different antigen variants. On the other hand, cross-reactivity in molecular binding interactions measures the extent to which different binding partners appear similar to a probe.

Exemplary binding characteristics of an antigen to its antibody and their effects on off rate vs. total dissociation time are disclosed in WO 2018/140157 and are reproduced below.

Examples of K_{off} rates:

| Antibody | Kₒₙ rates, M⁻¹ sec⁻¹ | K_{off} rates, sec⁻¹ | K_{D}, nM |
|---|---|---|---|
| a-TSH | 9.0 × 10⁴ | 1.1 × 10⁻³ | 12 |
| a-HGF | 9.5 × 10³ | 4.5 × 10⁻⁴ | 48 |
| a-EGF | 26.0 × 10⁴ | 1.3 × 10⁻³ | 51 |
| a-Leptin | 3.9 × 10³ | 1.3 × 10⁻⁴ | 33 |
| a-FADD | 2.1 × 10³ | 1.7 × 10⁻⁴ | 81 |

Effect of K_{off} rates on time to 90% dissociation

| K^{off} | Time to 90% Dissociation | |
|---|---|---|
| 0.1 | 23 | s |
| 1.E−02 | 3.8 | min |
| 1.E−03 | 38 | min |
| 1.E−04 | 16 | days |
| 1.E−05 | 160 | days |

The kinetic analysis of single molecules requires expensive equipment. Important engineering tradeoffs are considered when designing such a system. For example, when analyzing kinetic information, the amount of time averaging one can use to increase the system's signal to noise ratio is limited. Moreover, because one monitors the kinetic binding of molecules over time, the method is inherently slow unless molecules having very fast binding characteristics are used. In one example, it could take 5 to 10 minutes to observe several binding interactions towards positive kinetic fingerprinting of molecular interactions. In this situation, analyzing different molecules sequentially in time may not be practical and one must analyze different targets in parallel.

Achieving single molecule detection with an imaging system is not trivial, for example, because of the weak nature of fluorescence. There are difficult engineering tradeoffs to be made between illumination power, imaging area, imaging resolution, where all of these tradeoffs impact the cost and speed of a system and how many different targets can be imaged in parallel. This disclosure provides that using substrates that are designed to enhance the strength of a fluorescent signal allows a greater number of molecules to be analyzed in parallel. Using such substrates also allows the system to be faster and less expensive. Moreover, substrates that exhibit enhanced detection of signal arising from molecules located in a close proximity of the substrate's surface particularly enable the kinetic binding analysis of single molecules for target detection.

This disclosure provides methods for positively identifying interactions of a single molecule with its binding partner using a substrate that is tailored to enhance the fluorescent signal emitted by molecules located in close proximity of the substrate's surface. An example of this system is provided in FIG. 1.

This disclosure also relates to assays designed to minimize or eliminate the non-specific binding or noise utilizing single molecule detection technologies. The assay depends on the measurement on the assay surface of the probability of a molecule, preferably, a biomolecule, binding to all molecules, preferably, diverse array of biomolecules, including its binding partners. The interactions at the individual level of a molecule with its binding partners are observed on the assay surface for a certain period of time. This approach allows counting the number of molecules specifically bound to its binding partners located on the assay surface even in the presence of a much larger number of non-specific binding events due to the probing molecules interacting non-specifically with the capture surface. Such differentiation can be made based on the difference in the interaction or kinetic profile of a specific molecular interaction compared to that of a non-specific interaction. This minimization or elimination of the non-specific noise leads to a much lower limit of quantification.

Accordingly, certain embodiments of the invention provide methods of analyzing binding interactions between a probe and its target. The method comprises contacting the probe with the target that exhibits fast-off rate binding characteristics with the probe to generate binding interactions between probe and the target and detecting the binding interactions between the probe and the target by single molecule detection of probe at a plurality of locations, wherein the target is immobilized onto a substrate that is designed to enhance a fluorescence signal emitted from the probe that is located near the substrate's surface.

Thus, binding partners that emit a fluorescent signal or are conjugated to molecules that emit a fluorescent signal are allowed to contact a surface having immobilized thereon certain targets. The targets immobilized onto a surface can include molecules that specifically bind to the probes that emit the fluorescent signal or probes conjugated to molecules that emit a fluorescent signal. The probes interact transiently with many different targets immobilized on a surface, binding some targets for a long time before unbinding, while binding other targets for a shorter time, and still not at all binding some targets. In preferred embodiments, the transient binding events at the surface are imaged by a fluorescence microscope, such as total internal reflection fluorescence (TIRF) microscope.

In certain embodiments, a software program identifies all locations where repetitive binding of the probe molecule to the surface occurs. Then the software analyzes the kinetic behavior of each of these regions and identifies which regions can be attributed to the specific probe/target interactions of interest and which regions can be attributed to non-specific binding, which is not of interest. For example, certain probes reside at some spots (on) for longer periods compared to at other spots, which indicates that the probe binds at a different frequency to molecules at some spots compared to the molecules at other spots.

The following equation preferably characterizes transient binding:

K_{D}=K_{off}/Kₒₙ

Any suitable metric of the transient binding of the probe, including KD, K_{off} as well as other suitable measure can be used to generate a histogram. These include manipulations and any proxy of this equation.

The observed residence times of the binding of a probe with a target can be between about 1 nanosecond to about 10 minutes or more. Binding frequency can be measured by the total number of on- and off-events, i.e., transient events, observed at a specific spot in a time frame of about 1 nanosecond to about 10 min, 1 hour or one day. Different patterns of transient binding of the probe to the immobilized target biomolecules on the substrate's surface can be described by a combination of residence time and interaction frequency, the period of off-time, and/or signal intensity etc.

Accordingly, certain embodiments of the invention provide a method of determining a single molecule kinetic fingerprinting by enhancing fluorescent signal that indicates the binding interaction between one or more probes that emit a fluorescent signal and one or more targets, the method comprising:

a) immobilizing the one or more targets, for example, a capture antibody, on a substrate that is designed to enhance the fluorescent signal emitted by the one or more probes when located near the substrate's surface,

b) contacting the one or more probes with the one or more targets immobilized on the substrate,

c) detecting the interactions between the one or more probes and the one or more targets at single molecular level to determine the single molecule kinetic fingerprinting.

One or more targets can be immobilized on the substrate by conjugating to the substrate. For example, one or more targets (for example, nucleic acid targets) can be immobilized on the substrate using a hydrophilic self-assembled monolayer. One or more targets can be attached to a substrate prior to, or simultaneously with, surface passivation, for example, via a commercially available solution from SoluLinK. One or more targets can also be conjugated to a heterobifunctional PEG linker (e.g., PEG-4), which confers a benzaldehyde functionality. The substrate such as glass can be activated with for example, hydrazone functional groups, and one or more targets can be attached to the surface at low occupancy rates (<0.8 area %) through highly specific, efficient reactions between benzaldehyde derivatives of the one or more targets and surface hydrazone functionalities. Distribution of one or more targets over the substrate can be occupancy controlled by the concentration of the targets and the reaction time. Unoccupied surface regions can be passivated with monofunctional (benzaldehyde) PEG chains via the same coupling chemistry used in target immobilization. Additional examples of conjugating targets to a substrate are known in the art and certain such examples are described for example, in the PCT Publication Number WO 2018/140157, particularly, in paragraphs [0072] to [0079]. Such embodiments are within the purview of the invention. Other examples of conjugating targets to a substrate are described in: Bioconjugate Techniques, 3rd edition, 2013, Elsevier Inc., Ed: Greg T Hermanson; and Immobilization of Enzymes and Cells, 3rd edition, 2013, Ed: Jose M Guisan, Humana. The contents of these references are incorporated by reference in their entirety. Commercial methods of conjugating targets to a substrate are provided by ThermoFisher Scientific, Waltham, Mass., and use of such methods is within the purview of this invention. Moreover, methods of conjugating nucleic acids to substrates are used in nucleic acid analysis, for example, in next generation sequences and microarray analysis. Use of such methods is also within the purview of this invention.

One or more targets can also be immobilized on the substrate via specific binding to entities that are conjugated to the substrate. For example, a capture antibody can be immobilized on the surface. In another example, one or more targets can be conjugated to biotin that binds via specific binding to streptavidin to entities conjugated to the substrate and containing streptavidin. Additional examples of such binding pairs that can be used to immobilize one or more targets to entities immobilized on the substrate are known in the art and such embodiments are within the purview of the invention.

One or more targets can be obtained from a sample, for example, a biological sample. Such biological sample can be treated with a substrate having conjugated thereto one or more entities that specifically bind to one or more targets in the sample. The locations on the substrate of the one or more entities that specifically bind to the one or more targets are known. Once the one or more targets from the biological sample are bound to the entities on the substrate, unbound molecules from the biological sample can be washed from the substrate and the substrate can then be contacted with one or more probes to study the binding interactions between the one or more probes and the one or more targets immobilized on the substrate.

Several examples are known in the art of surfaces that are designed to enhance a fluorescence signal emitted from molecules located near the substrate's surface. For example, examples of surfaces that enhance a fluorescence signal emitted from fluorescent molecules located near the substrate's surface are disclosed the following documents (each of which is hereby incorporated by reference in its entirety): European Patents Numbers: EP2257790 (U.S. Pat. No. 8,679,855) and EP2110658 (U.S. Pat. No. 8,421,036); U.S. Pat. Nos. 9,459,212, 9,464,988, 8,993,339, and 9,709,538; and Unite States Patent Application Publication Numbers: 20150377783, 20170152549, 20190017934, 20190262947, 20170168048, 20150338345, 20140374621, 20140323323, 20140256593, 20130094021, 20120058697, 20110269644, 20100159614, 20100009862, 20090117006, 20080166270, 20020132376, and 20020010279. In certain embodiments, the substrate comprises prism based Surface Plasmon Resonance architectures. In preferred embodiments, the substrate comprises metallic grating structures, photonic crystal structures, nano patterned surfaces, or nano structured surfaces. Additional examples substrates that are designed to enhance fluorescent signals emitted from molecules located near the substrate's surface are known in the art and such embodiments are within the purview of the invention.

In certain embodiments, the probes are designed to enhance a fluorescence signal emitted from the probes located near a substrate's surface. For example, the probes can be conjugated to nanoparticles, such as fluorescent enhancing nanoparticles or beads (gold or silver nanoparticles or beads), that enhance a fluorescence signal emitted from the probes when located near the substrate's surface.

The step of contacting the one or more probes with the one or more targets immobilized on the substrate is conducted under conditions that allow the binding between the one or more probes and the one or more targets. Typically, such conditions include appropriate buffer, appropriate temperature, and appropriate length of time. A person of ordinary skill in the art can determine appropriate conditions for a given set of one or more probes and one or more targets.

The step of detecting the interactions between the one or more probes and the one or more targets at single molecular level comprises detecting the presence of the one or more probes at the locations on the substrate where the one or more targets are immobilized. Several methods are known in the art for detecting in real time a fluorescent signal at molecular level, for example, arising from molecules located at known positions on a substrate. Certain examples of detecting in real time fluorescent signals at a single molecular level are reviewed and described by Croop et al. (2019), WIREs Systems Biology and Medicine, Vol. 11, Issue. 4, e1445. The Croop et al. publication is incorporated herein by reference in its entirety. Additional examples of detecting in real time fluorescent signals at a single molecular level are known in the art and such embodiments are within the purview of the invention.

In preferred embodiments, the transient interactions between at least one of the one or more probes and at least one of the one or more targets exhibit fast-off rate binding characteristics. For example, the transient interactions between at least one of the one or more probes and at least one of the one or more targets have an observed residence time of about 10 minutes to about 1 nanosecond.

Detecting the interactions between the one or more probes and the one or more targets and correlating the interactions to determine the binding characteristics of the one or more probes with the one or more targets can be used to differentiate specific binding interactions between the one or more probes and the one or more targets from the non-specific binding of the probe molecule to the surface around the capture molecule. To differentiate between the specific and non-specific bindings, several binding characteristics are considered, for example, the total number of on and off events at a particular target molecule and the number of transient interactions.

What is claimed is:

1. A method for determining a single molecule kinetic fingerprinting by enhancing fluorescent signal that indicates the binding interaction between one or more probes that emit a fluorescent signal and one or more targets, the method comprising:
a) immobilizing the one or more targets on a substrate that enhances the fluorescent signal emitted by the one or more probes when located near the substrate's surface,
b) contacting the one or more probes with the one or more targets immobilized on the substrate,
c) detecting the interactions between the one or more probes and the one or more targets at single molecular level to determine the single molecule kinetic fingerprinting.

2. The method of claim 1, wherein the one or more targets are immobilized on the substrate by conjugating to the substrate or are immobilized on the substrate via specific binding of the one or more targets to the entities that are conjugated to the substrate.

3. The method of claim 1, wherein the one or more targets are obtained from a biological sample.

4. The method of claim 1, wherein the substrate is designed to enhance a fluorescence signal emitted from the one or more probes when located near the substrate's surface and/or the probes is conjugated to fluorescent enhancing nanoparticles or beads that enhance the fluorescence signal emitted from the probes when located near the substrate's surface.

5. The method of claim 1, wherein the substrate comprises prism based surface plasmon resonance architecture, metallic grating structure, photonic crystal structure, nano patterned surface, or nano structured surface.

6. The method of claim 1, comprising detecting in real time the interactions between the one or more probes and the one or more targets at single molecular level to determine the single molecule kinetic fingerprinting.

7. The method of claim 1, wherein detecting the interactions between the one or more probes and the one or more targets at single molecular level to determine the single molecule kinetic fingerprinting comprises visualizing the presence of the one or more probes at the locations on the substrate where the one or more targets are immobilized.

8. The method of claim 1, wherein the transient interactions between at least one of the one or more probes and at least one of the one or more targets exhibit fast-off rate binding.

9. The method of claim 1, wherein the transient interactions between at least one of the one or more probes and at least one of the one or more targets have an observed residence time of about 1 nanosecond to about 10 minutes.

10. The method of claim 1, further correlating the interactions between the one or more probes and the one or more targets at single molecular level to determine the binding characteristics of the one or more probes with the one or more targets.

11. The method of claim 1, further comprising correlating the interactions between the one or more probes and the one or more targets to differentiate specific binding interactions between the one or more probes and the one or more targets from the non-specific binding interactions between the one or more probes and the one or more targets and/or with areas around the target.

12. The method of claim 1, comprising detecting the interactions between the one or more probes and the one or more targets at single molecular level to determine the single molecule kinetic fingerprinting at a plurality of locations.

13. The method of claim 1, wherein detecting the interactions between the one or more probes and the one or more targets comprises measuring the total number of transient events.

14. The method of claim 1, wherein the binding interactions between the one or more probes and the one or more targets induce the emission of a fluorescent signal via fluorescence resonance energy transfer between one or more donor fluorophores that are conjugated to the one or more probes and one or more acceptor probes that are conjugated to the one or more targets.

15. The method of claim 1, wherein the binding interactions between the one or more probes and the one or more targets induce emission of a fluorescent signal via fluorescence resonance energy transfer between one or more donor fluorophores that are conjugated to the one or more targets and one or more acceptor probes that are conjugated to the one or more probes.
